(19) United States
(12) Patent Application Publication
(10) Pub. No.: US 2023/0031925 A1
(43) Pub. Date: Feb. 2, 2023

(54) FULL-DUPLEX IPG SYSTEM AND ELECTRO-OPTICAL PERCUTANEOUS LEAD

(71) Applicant: Wavegate Corporation, Lake Charles, LA (US)

(72) Inventors: Erich W. Wolf, II, Lake Charles, LA (US); Dennis Patrick O'Neal, Coppell, TX (US)

(73) Assignee: Wavegate Corporation, Lake Charles, LA (US)

(21) Appl. No.: 17/815,482

(22) Filed: Jul. 27, 2022

Related U.S. Application Data
(60) Provisional application No. 63/203,649, filed on Jul. 27, 2021.

(51) Int. Cl. A61N 5/06; A61N 5/067

(52) U.S. Cl. CPC A61N 5/0601; A61N 5/067; A61N 2005/0626; A61N 2005/063; A61N 2005/0666; A61N 2005/0663; A61N 2005/0659

(57) ABSTRACT

The invention provides an IPG and lead configuration which boasts both a novel optical folding assembly and an optical processor assembly which offers the advantages of low heat generation and compact package size. The surgical leads provided offer additional advantages over the prior art including integral formation of optical and electrical components in a compact size. The invention further provides processing advantages which measure and compensate for degradation in the optical system over time.

### CROSS-REFERENCE TO RELATED APPLICATIONS

This application claims priority benefit from U.S. Provisional Application No. 63/203,649 filed on Jul. 27, 2021. The patent application identified above is incorporated here by reference in its entirety to provide continuity of disclosure.

### FIELD OF THE INVENTION

The present invention relates to an improved implantable pulse generator (“IPG”) and header combination using optical reflectometry in spinal cord stimulation (“SCS”).

### BACKGROUND OF THE INVENTION

Chronic pain may arise from a variety of conditions, most notably from nerve injury as in the case of neuropathic pain, or from chronic stimulation of mechanical nociceptors such as with spinal pain. Functional ability may be severely impacted by pain, which often is refractory to pharmacological and surgical treatment. In such cases, SCS can be an effective treatment for pain by modulating physiological transmission of pain signals from the periphery to the brain. This may be achieved by applying electrical impulses to the spinal cord via an electrode array implanted adjacent the spinal canal.

Referring to FIGS. 1, 2 and 3, a typical IPG system of the prior art will be described. Spinal column 1 is shown to have a number of vertebrae, categorized into four sections or types: lumbar vertebrae 2, thoracic vertebrae 3, cervical vertebrae 4 and sacral vertebrae 5. Cervical vertebrae 4 include the 1st cervical vertebra (C1) through the 7th cervical vertebra (C7). Just below the 7th cervical vertebra is the first of twelve thoracic vertebrae 3 including the 1st thoracic vertebra (T1) through the 12th thoracic vertebra (T12). Just below the 12th thoracic vertebrae 3, are five lumbar vertebrae 2 including the 1st lumbar vertebra (L1) through the 5th lumbar vertebra (L5), the 5th lumbar vertebra being attached to sacral vertebrae 5 (S1 to S5), sacral vertebrae 5 being naturally fused together in the adult.

Representative vertebra 10, a thoracic vertebra, is shown to have a number of notable features which are in general shared with lumbar vertebrae 2 and cervical vertebrae 4. The thick oval segment of bone forming the anterior aspect of vertebra 10 is vertebral body 12. Vertebral body 12 is attached to bony vertebral arch 13 through which spinal nerves 11 run. Vertebral arch 13, forming the posterior of vertebra 10, is comprised of two pedicles 14, which are short stout processes that extend from the sides of vertebral body 12 and bilateral laminae 15. The broad flat plates that project from pedicles 14 join in a triangle to form a hollow archway, spinal canal 16. Spinous process 17 protrudes from the junction of bilateral laminae 15. Transverse processes 18 project from the junction of pedicles 14 and bilateral laminae 15. The structures of the vertebral arch protect spinal cord 20 and spinal nerves 11 that run through the spinal canal.

Surrounding spinal cord 20 is dura 21 that contains cerebrospinal fluid (CSF) 22. Epidural space 24 is the space within the spinal canal lying outside the dura.

One or more electrodes 30 are positioned in epidural space 24 between dura 21 and the walls of spinal canal 16 towards the dorsal aspect of the spinal canal nearest bilateral laminae 15 and spinous process 17. Electrode 30 has electrode leads 31 which are connected to IPG 32 and controller 33.

IPG 32 provides the electrical stimulation in the form of current pulses to the spinal cord through lead 31 to electrode 30. The pulses generate an electric field. The electric field impinges on targeted neurons of the spinal cord and disrupts the perception of pain. The amplitude of the electrical field is critical to success of spinal cord stimulation. An inadequate electric field will fail to depolarize the targeted neurons, rendering the treatment ineffective. An excess electric field stimulates neighboring cell populations which results in a noxious stimulation.

Establishing a consistent, therapeutic, and non-noxious level of stimulation is predicated upon establishing an ideal current density within the spinal cord's targeted neurons. Fundamentally, this should be a simple matter of establishing an optimal electrode current given the local bulk conductivity of the surrounding tissues. But in practice, the optimal electrode current changes as a function of patient position and activity due to motion of the spinal cord as the spinal cord floats in cerebrospinal fluid within the spinal canal. Significant changes in distance between the epidural electrode array and the targeted spinal cord neurons have been shown to occur. Consequently, optimal stimulation requires dynamic adjustment of the electrode stimulating current as a function of distance between the electrode array and the spinal cord.

Dynamic modulation of spinal cord stimulator electrode current as a function of distance between the electrode array and the spinal cord thus has several benefits. Excess stimulation current can be avoided, thus reducing the prospects of noxious stimulation and potentially reducing device power consumption. Inadequate stimulation current can also be avoided, thus eliminating periods of compromised therapeutic efficacy.

Dynamic modulation of electrode current can be controlled through the use of optical reflectometry to determine the thickness of the dorsal cerebrospinal fluid (dCSF) column between the spinal cord and the electrode array. An optical signal is transmitted into the surrounding tissue and collected by a sensor to calculate the approximate distance between the electrode and the spinal cord. The stimulus magnitude is modified accordingly to provide the optimal current for pain relief. Examples of this technology are shown in U.S. Pat. Nos. 10,035,019 and 9,656,097, both to Wolf II, and both incorporated herein by reference.

One challenge, to IPG implantation and usage is package size. Long-term survival of the IPG in the in vivo environment, mitigates that the package size be as small as possible to reduce rejection rates and to lower scarring times. A small package size also decreases the possibility of migration of the package and the surgical leads which emanate from it.

Another challenge to IPG systems is proper interpretation of the reflected optical signal. Half duplex systems of the prior art require that multiple surgical leads be precisely placed by the targeted neurons. Such placement during surgery is difficult. The problem is further exacerbated by lead migration, which may disalign the fibers and degrade optical feedback which controls modulation of the stimulation signal.

Yet another challenge to IPG systems is power constraints and heat generation. Power usage of the IPG must be kept to a minimum in order to assure long term battery life of the IPG package. Further, heat generation in the in vivo environment must be minimized to prevent rejection. Therefore, power consumption and heat generation both must be as low as possible.

Yet another challenge to optical IPG systems is Fresnel reflections. Fresnel reflections occur at all optical interfaces of the lead and through fiber and can be assumed to account for about 4% signal loss per optical interface. Interface losses are generally constant over time. Fiber degradation caused by bending also produces Fresnel losses. However, fiber degradation losses are not constant and change over time. Optical losses effect the accuracy of the optical feedback which controls the stimulation signal.

Another challenge to optical IPG systems is that laser output changes due to power variations and due to part age, thereby altering the characteristics of the optical system over time. The changes in the optical system reduce the effectiveness of the stimulation signal control and so reduce the efficacy of the stimulation signal.

The prior art has attempted to address these challenges in a number of ways, yet all have fallen short.

For example, U.S. Pat. No. 9,656,097 to Wo/f, II describe a full duplex IPG lead, which allows both a transmit ray and receive ray to travel down the same fiber. However, Wolf discloses use of a circulator to separate the two rays. A circulator is not practical because of the size of the circulator package and because of the optical losses incurred.

As another example, U.S. Pat. No. 7,742,817 to Malinowski, et al. describes an IPG with connectors for electrical leads and an epoxy coating for biocompatibility. However, Malinowski does not disclose the use of optical feedback to achieve proper stimulation signal strength.

As another example, U.S. Publication No. 2021/0001114 to Wo/f, II, discloses coupling of an optical lead to an IPG header adjacent a ruby passthrough window, vertically aligned with a photo detector. However, Wolf fails to disclose a way to recognize or compensate for optical system degradation over time.

U.S. Publication No. 2018/0154152 to Chabrol discloses a system for deep brain stimulation using a probe with stimulation electrodes and a light emitting optical fiber. However, Chabrol fails to address using the light signal to control a stimulation signal to the spinal cord. Chabrol also fails to recognize optical signal degradation over time or a way to test for resulting losses.

Deficiencies exist in the prior art related to power usage constraints, heat generation, and maintaining accurate optical feedback over time. Thus, there is a need in the art for an improved IPG including header orientation, connectors, leads and electrodes which provide a stable optical signal while tracking optical signal degradation due to Fresnel reflections and reducing power consumption and heat generation.

### BRIEF DESCRIPTION OF THE DRAWINGS

In the detailed description of the preferred embodiments presented below, reference is made to the accompanying drawings.

FIG. 1 is a side view of the human spine showing the approximate position of a percutaneous lead and IPG for spinal cord stimulation.

FIG. 2 shows an axial view of a thoracic vertebra indicating the position of the spinal cord and a percutaneous lead pair.

FIG. 3 shows a sagittal cross-sectional view of the human spine showing the approximate position of a percutaneous lead.

FIG. 4 shows a schematic diagram of an IPG system of a preferred embodiment.

FIG. 5 is an isometric view of a preferred IPG device.

FIG. 6 is an exploded isometric view of a preferred IPG device.

FIG. 7 is an exploded isometric view of a preferred IPG case.

FIG. 8 is an exploded isometric view of a preferred IPG header.

FIG. 9 is an exploded isometric view of a preferred die stack.

FIG. 10 is a partial cross-section view of a preferred header assembly.

FIG. 11 is an isometric view of a lead assembly of a preferred embodiment.

FIG. 12 is a cross-section view of a lead assembly of a preferred embodiment.

FIG. 13 is a flowchart of a preferred control program for operation of the IPG.

FIG. 14 is a flowchart of a preferred method of Fresnel compensation.

FIG. 15 is a flowchart of a preferred method of normalizing source emission.

FIG. 16 is a flowchart of a preferred method of correcting for source power variation.

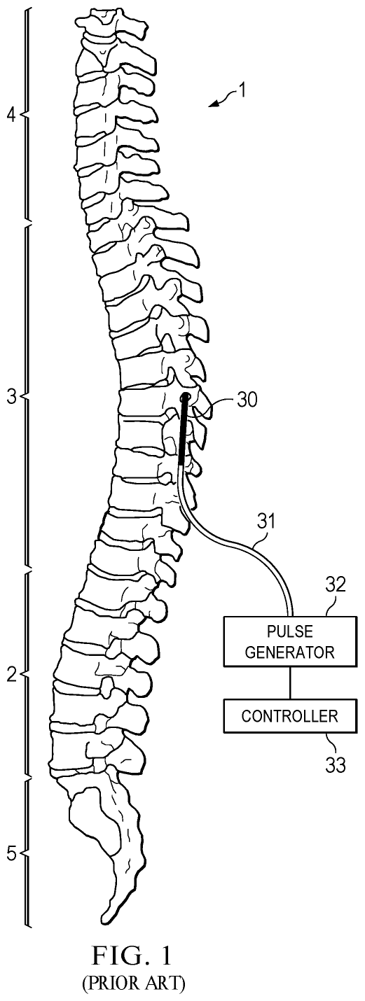

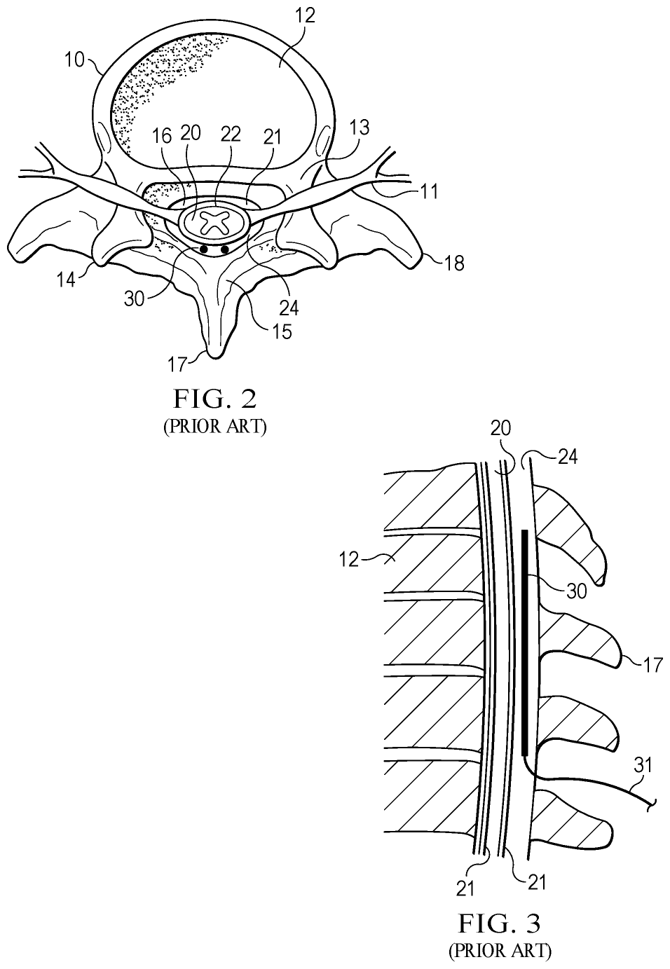

### DETAILED DESCRIPTION OF THE INVENTION

In the description that follows, like parts are marked throughout the specification and figures for the same numerals. The figures are not necessarily drawn to scale and may be shown in exaggerated or generalized form in the interest of clarity and conciseness. Unless otherwise noted, all tolerances and uses of the term “about” indicate plus or minus 5%.

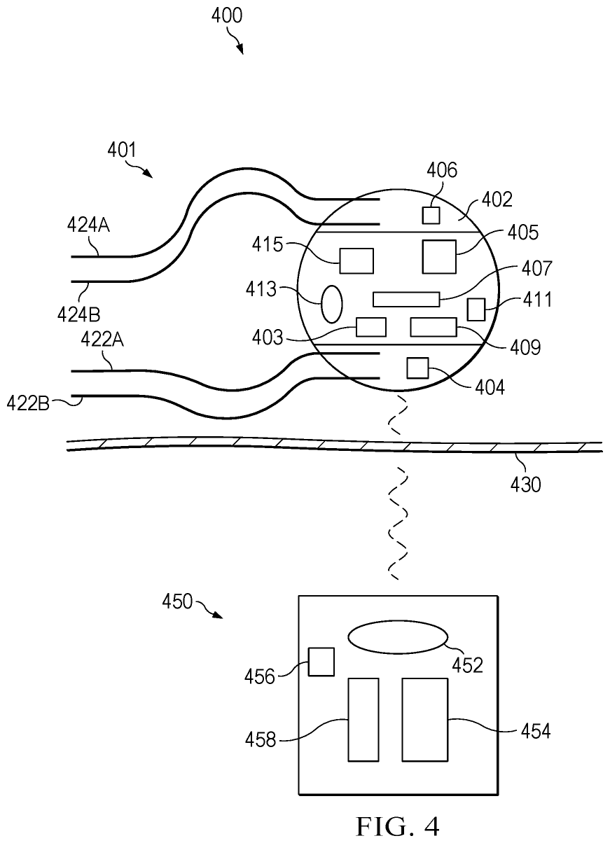

Referring then to FIG. 4, preferred embodiment of stimulation system 400 will be further described. Stimulation system 400 further comprises IPG 401 in operative communication with controller 450.

IPG 401 is housed in hermetically sealed composite case 402, as will be further described. Composite case 402 houses the operative components of the IPG and serves to anchor leads 422A, 422B, 424A and 424B, as will be further described.

The operative components of the system comprise optical folding assembly 406, optically aligned with leads 424A and 424B. Similarly, the operative components include optical folding assembly 404, optically aligned with leads 422A and 422B. Optical folding assembly 406 sends and receives optical signals from leads 424A and 424B to be interpreted by optical signal processor 405. Likewise, optical folding assembly 404 sends and receives optical signals from leads 422A and 422B to be interpreted by optical signal processor 403. Optical signal processor 405 and optical signal processor 403 are operatively connected to main processor 407, which controls the functions of the IPG, as will be further described. Main processor 407 is operatively connected to signal generator 409, which generates electrical stimulation signals which are transmitted through leads 424A, 424B, 422A and 422B to the spinal cord to targeted nerve populations, as will be further described. Communications circuit 411 is also operatively connected to main processor 407. Main processor 407 receives programming instructions and control signals from the communications circuit, as will be further described.

IPG 401 includes battery 415. Battery 415 is operatively connected to all the electrical components of the system. Battery 415 receives recharging current from induction coil 413, as will be further described.

IPG 401 is surgically implanted beneath dermis 430.

Controller 450 exists outside dermis 430. Controller 450 includes main processor 454, which controls the functions of the controller. Main processor 454 is connected to I/O keyboard and display unit 458, which is fixed in the exterior casing. Main processor 454 is operatively connected to communications circuit 456. Communications circuit 456 is wirelessly connected to communications circuit 411. Main processor 454 includes sufficient memory to receive instructions from I/O keyboard and display unit 458 and transfer them through communications circuit 456 and communications circuit 411 to main processor 407 for controlling the operation of IPG 401. Main processor 454 is further operatively connected to induction coil 452. Induction coil 452 is inductively coupled to induction coil 413 which transfers power for the charging of the battery.

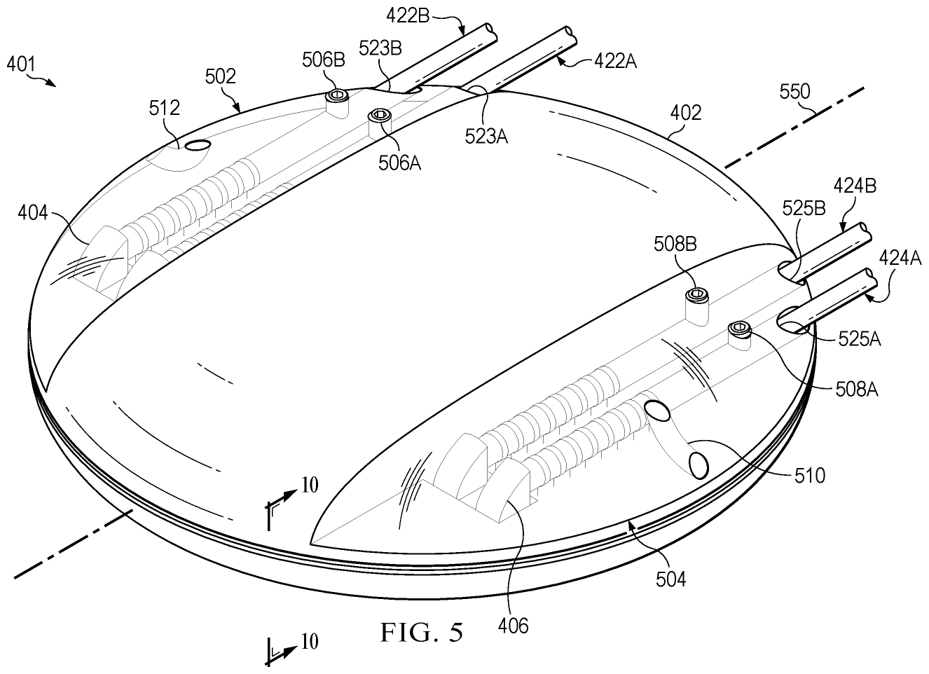

Referring then to FIG. 5, IPG 401 will be further described.

Composite case 402 mechanically supports header assembly 502 and header assembly 504, which are both positioned on opposite sides of the IPG, parallel to a single lateral axis 550. Positioning the header assemblies at opposite sides of the case is important because it improves the dispersion of the heat generated by the lasers, as will be further described. Each header assembly preferably is manufactured from a transparent epoxy resin which serves to fix the optical and electrical components in place.

Header assembly 502 includes optical folding assembly 404. Optical folding assembly 404 is optically aligned with lead retainer holes 523A and 523B. Leads 422A and 422B are positioned in and optically aligned by lead retainer holes 523A and 523B. Leads 422A and 422B are removably secured in the header by virtue of set screws 506A and 506B, respectively. The leads abut optical folding assembly 404, as will be further described.

Header assembly 502 further comprises tie down portal 512, for use in securing the IPG to the fascia during surgery.

Header assembly 504 includes optical folding assembly 406. Optical folding assembly 406 is optically aligned with lead retainer holes 525A and 525B. Leads 424A and 424B are positioned in and optically aligned by lead retainer holes 525A and 525B. Leads 424A and 424B are removably secured in the header by virtue of set screws 508A and 508B, respectively. The leads abut optical folding assembly 406, as will be further described.

Header assembly 504 further comprises tie down portal 510, for use in securing the IPG to the fascia during surgery.

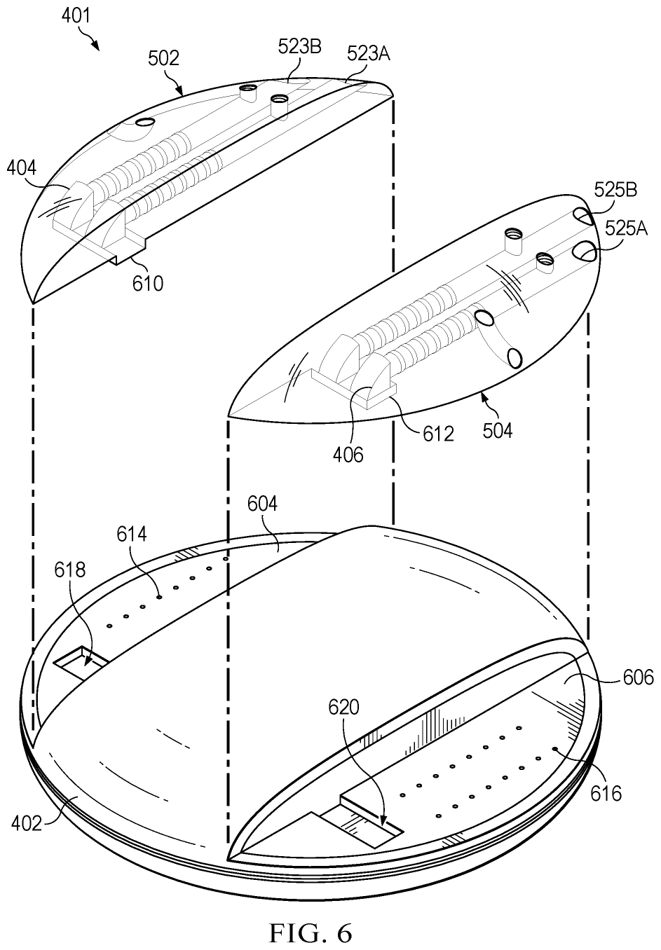

Referring then to FIG. 6, IPG 401 will be further described.

Composite case 402 further comprises header bay 604 and header bay 606. Each header bay is an angular indention in the composite case. The header bays are generally parallel and diametrically opposed. Header bay 604 houses header assembly 502. Likewise, header bay 606 houses header assembly 504.

Header bay 604 includes rectangular receiving window 618, and electrical pass-through holes 614. The receiving window and the electrical pass-through holes allow for connections between header assembly 502 and the internal components of the IPG.

Likewise, header bay 606 includes rectangular receiving window 620 and electrical pass-through holes 616 for connection of the components of header assembly 504 with the internal components of the IPG.

Header assembly 502 includes integrally formed positioning block 610. Positioning block 610 fits within receiving window 618 and optically aligns the header bay with the optical components in the case. Header assembly 504 includes integrally formed positioning block 612. Positioning block 612 fits within receiving window 620 and optically aligns the header bay with the optical components in the case.

Composite case 402 including both headers forms an elliptical surface of revolution as described in U.S. Publication No. 2021/0001114 to Wolf, incorporated herein by reference.

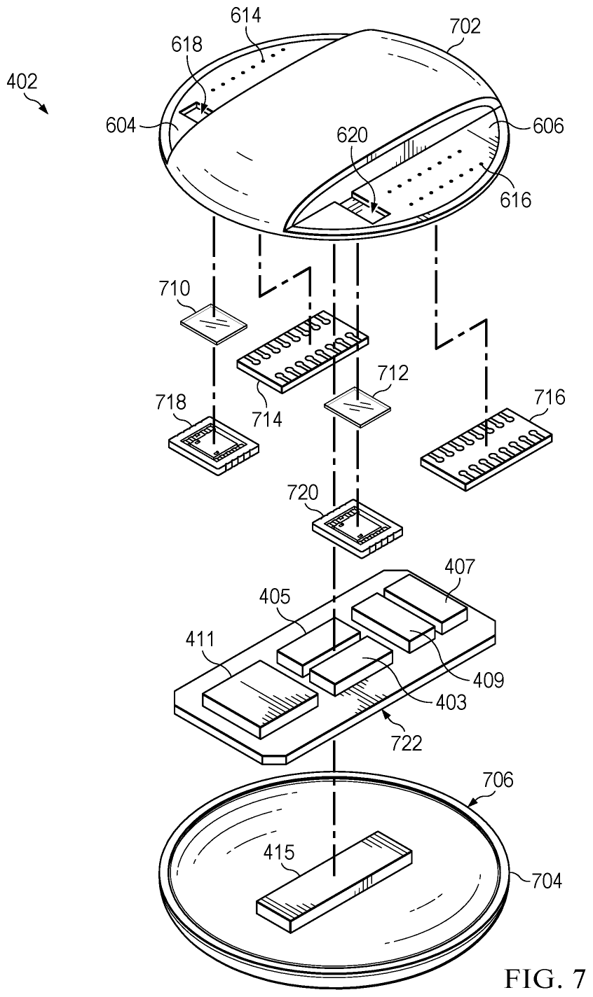

Referring to FIG. 7, composite case 402 will be further described.

Composite case 402 includes top section 702 joined to bottom section 704. Top section 702 is preferably a hollow shell manufactured from a titanium alloy. The titanium alloy is relatively easily machined and can hold tolerances sufficient to allow proper alignment of headers and optical components, as will be further described. Bottom section 704 is preferably a hollow shell comprised of a ceramic material. The ceramic material facilitates radio communication between the communication circuits and inductive coupling between the inductive coils. The combination of a ceramic material with a metallic material for the case is also important because the heat generated from the IPG is directed toward the metallic section of the case. The metallic section of the case may be positioned toward the dermis during surgery, thus allowing the IPG to be positioned for superior heat dispersion while in use.

Optical window 710 is positioned adjacent top section 702 and centered beneath receiving window 618. Likewise, optical window 712 is positioned adjacent top section 702 and centered beneath receiving window 620. Both optical windows are preferably welded and sealed to the underside of the top section.

Die stack 718 is functionally positioned beneath optical window 710 and adjacent optical signal processor 405, as will be further described. Die stack 720 is functionally positioned beneath optical window 712 and adjacent optical signal processor 403, as will be further described.

Connector card 714 is positioned adjacent top section 702 and directly beneath electrical pass-through holes 614. Connector card 716 is positioned adjacent top section 702 directly beneath electrical pass-through holes 616. Connector card 714 and connector card 716 are electrically connected to contacts in the headers and main processor 407, and allow for transmission of stimulation current to the leads, as will be further described.

Composite case 402 houses processor card 722. Processor card 722 structurally and electrically connects main processor 407 to optical signal processors 405 and 403, signal generator 409 and communications circuit 411. In a preferred embodiment, main processor 407 is Part No. MSP430, available from Texas Instruments of Dallas, Tex. Signal generator 409 is available under the tradename Saturn, available from Cactus Semiconductor. Communications circuit 411 is preferably Part No. ZL70103, available from Microsemi Corporation of Aliso Viejo, Calif. Optical signal processors 403 and 405 are both preferably Part No. ADPD4100, available from Analog Devices of Wilmington, Mass.

Composite case 402 further houses battery 415 which is electrically connected to processor card 722. The operational components of the IPG are preferably positioned adjacent top section 702 and held in place by an epoxy encapsulation. After encapsulation, bottom section 704 is hermetically sealed to top section 702 by weldment 706.

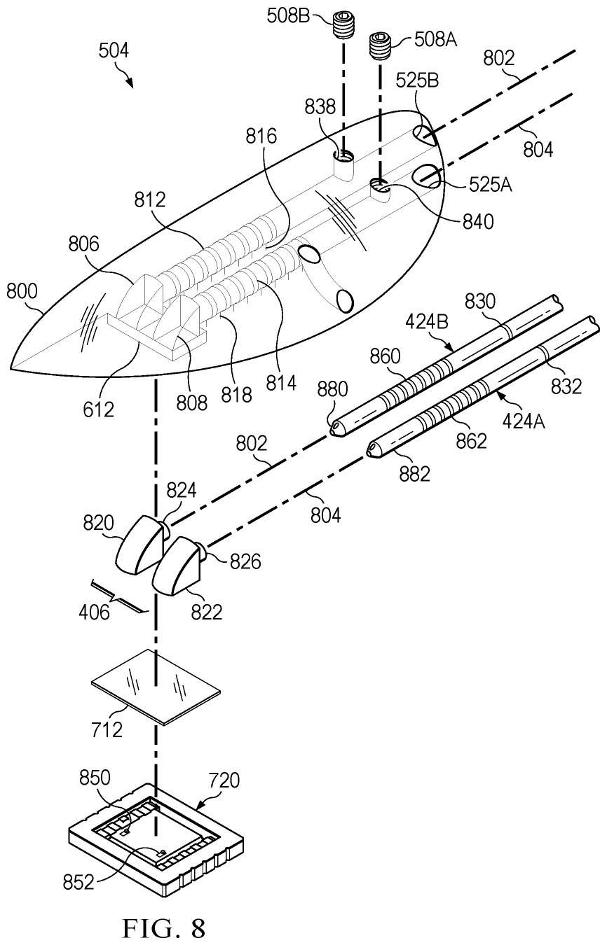

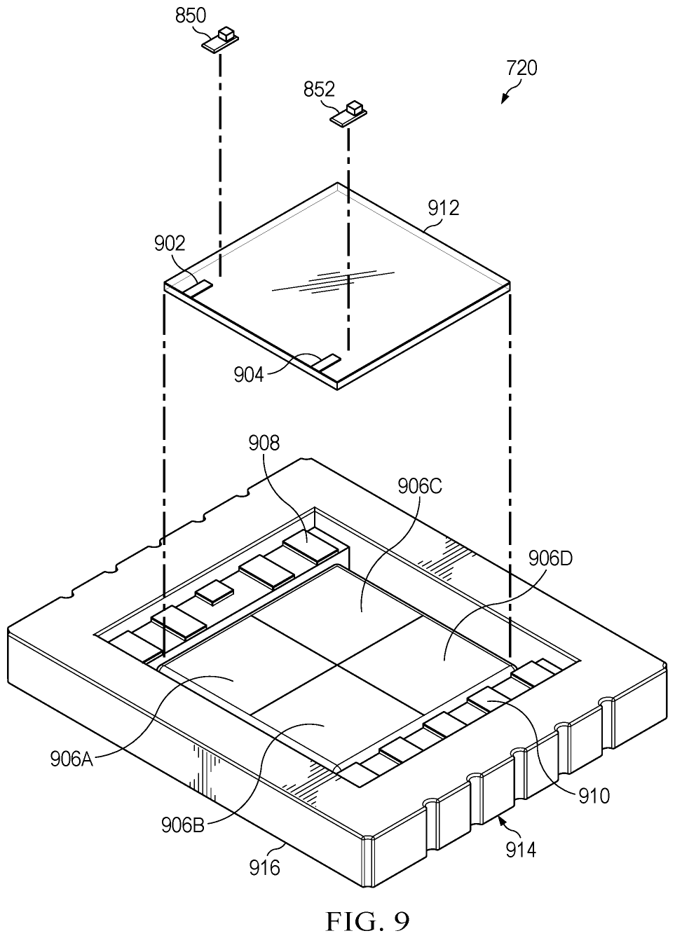

Referring then to FIG. 8, header assembly 504 will be further described. It should be understood that header assembly 502 is structurally and functionally identical to header assembly 504, with the exception of the components being in reversed positions. Only header assembly 504 will be described in detail here as an example.

Header assembly 504 includes header body 800. Header body 800 preferably is manufactured from a transparent epoxy resin, or acrylic plastic, cast and machined to tolerance. Header body 800 is formed in a semi-ellipsoidal shape, sufficient to fit seamlessly within header bay 606 and comport with the surface of revolution formed by top section 702 and bottom section 704.

Header body 800 includes lead retainer holes 525A and 525B. Lead retainer hole 525A includes central optical axis 804. Lead retainer hole 525B includes central optical axis 802. Optical axis 802 and optical axis 804, preferably, are generally parallel.

Lead retainer hole 525A includes metallic toroidal contacts 814. In a preferred embodiment, eight toroidal contacts are included, each individually addressable by the main processor. Toroidal contacts 814 are each connected to a lead wire 818. Lead wires 818 are positioned to intersect electrical pass-through holes 616 and be connected to the connector card which further connects them to the main processor. Lead retainer hole 525A terminates in cavity 808, adjacent positioning block 612, as will be further described.

Lead retainer hole 525B includes metallic toroidal contacts 812. In a preferred embodiment, eight toroidal contacts are included, each individually addressable by the main processor. Toroidal contacts 812 are each connected to a lead wire 816. Lead wires 816 are positioned to intersect electrical pass-through holes 616 and be connected to the connector card which further connects them to the main processor. Lead retainer hole 525B terminates in cavity 806 adjacent positioning block 612, as will be further described.

Lead retainer hole 525A includes threaded perpendicular hole 840. When lead 424A is positioned within lead retainer hole 525A, set screw 508A is positioned in threaded perpendicular hole 840 directly adjacent anchor ring 832 and secures lead 424A in lead retainer hole 525A.

Likewise, lead retainer hole 525B includes threaded perpendicular hole 838. Set screw 508B is positioned within threaded perpendicular hole 838 directly adjacent to lead anchor ring 830. Set screw 508B is advanced to contact lead anchor ring 830 and holds lead 424B in lead retainer hole 525B.

Optical folding assembly 406 further comprises parabolic redirector 820 and parabolic redirector 822. Parabolic redirector 820 includes integral lens 824. Parabolic redirector 822 includes integral lens 826. Parabolic redirector 820 and parabolic redirector 822 are rigidly secured in cavity 806 and cavity 808, respectively, in contact with and adjacent to optical window 712, as will be further described. In a preferred embodiment, the parabolic redirectors are cast in place in the header. Integral lens 824 is optically aligned with optical axis 802. Integral lens 826 is optically aligned with optical axis 804.

Die stack 720 is positioned directly below and in contact with optical window 712. Die stack 720 includes vertical cavity surface emitting laser (“VCSEL”) 850 and VCSEL 852, as will be further described. In each case, the VCSELs are capable of emitting light in an intrinsic wavelength range, but preferably in the range of about 400-810 nanometers or from blue (about 400 nanometers to about 500 nanometers), to green (about 520 nanometers to about 532 nanometers) to near IR (about 700 nanometers to about 810 nanometers). In a preferred embodiment, each VCSEL is Part No. V00146, available from Vixar, Inc. of Plymouth, Minnesota. Each laser produces about 10 milliwatts in the range of near IR and about 5 milliwatts in the blue range. Other lasers may be utilized in the wavelength range of about 400-580 nanometers (blue, aqua, green and yellow) as well as other visible ranges. VCSEL 850 is positioned to emit light vertically toward the central vertical optical axis of parabolic redirector 820. Likewise, VCSEL 852 is positioned to emit light vertically toward the central vertical optical axis of parabolic redirector 822.

Toroidal contacts 812 are designed to engage and electrically contact rings 860 of lead 424B, as will be further described. Toroidal contacts 814 are designed to engage and electrically contact rings 862 of lead 424A, as will be further described.

Lead 424B is positioned to align with optical axis 802 by lead retainer hole 525B. Lead 424A is positioned to align with optical axis 804 by lead retainer hole 525A.

Lead 424B terminates in collet 880, as will be further described. Lead 424A terminates in collet 882, as will be further described. When assembled, lead 424B and collet 880 are held adjacent to integral lens 824 by set screw 508B. Lead 424A and collet 882 are held adjacent integral lens 826 by set screw 508A. An index matching gel is provided to minimize Fresnel reflections between the lenses and the leads.

Referring in to FIG. 9, die stack 720 will be further described. It should be understood that die stack 718 and die stack 720 are functionally and structurally identical. Only die stack 720 will be described in detail.

Die stack 720 includes photodiode package 914. In a preferred embodiment, photodiode package 914 is Part No. S5980-09(ESI), available from Hamamatsu Photonics K.K. of Shizuoka, Japan.

The preferred photodiode package has four photodiodes. In one embodiment, all four photodiodes are used as photoreceivers. In another embodiment, two photodiodes 906A and 906B may be used as photoreceivers, one for each lead, and two photodiodes 906C and 906D, are used as monitor photodiodes one for each laser. The two monitor photodiodes act as output monitors to normalize the laser emission and correct for source power variations over time, as will be further described.

Photodiodes 906A, 906B, 906C and 906D are recessed and fixed in housing 916. Housing 916 is preferably a ceramic composite. Housing 916 is held in position in the case by epoxy encapsulation and rigidly fixes the position of the photodiodes. Each of the photodiodes typically provides a sensitivity of about 0.72 A/W. Photodiodes 906A, 906B, 906C and 906D are bounded by electrical contacts 908 and 910. These contacts are operatively connected to the appropriate optical signal processor by flexible cables (not shown). Cover plate 912 is positioned adjacent to and in contact with the four photodiodes. In a preferred embodiment, cover plate 912 is formed of a crystal glass polished to have optically parallel opposing faces. Cover plate 912 includes gold trace 902 and gold trace 904. The gold traces are preferably deposited on the glass surface opposite the photodiodes using photolithography or vapor deposition. VCSEL 850 is rigidly fixed to cover plate 912 adjacent to and in electrical contact with gold trace 902. VCSEL 852 is rigidly fixed to optical window 712 adjacent to and in electrical contact with gold trace 904. The gold traces provide power to the VCSELs and allow the main processor to select which wavelength laser to activate, as will be further described. Gold trace 902, gold trace 904, and contacts 908 and 910 are electrically connected to the appropriate optical signal processor and main processor 407 by flexible cables (not shown).

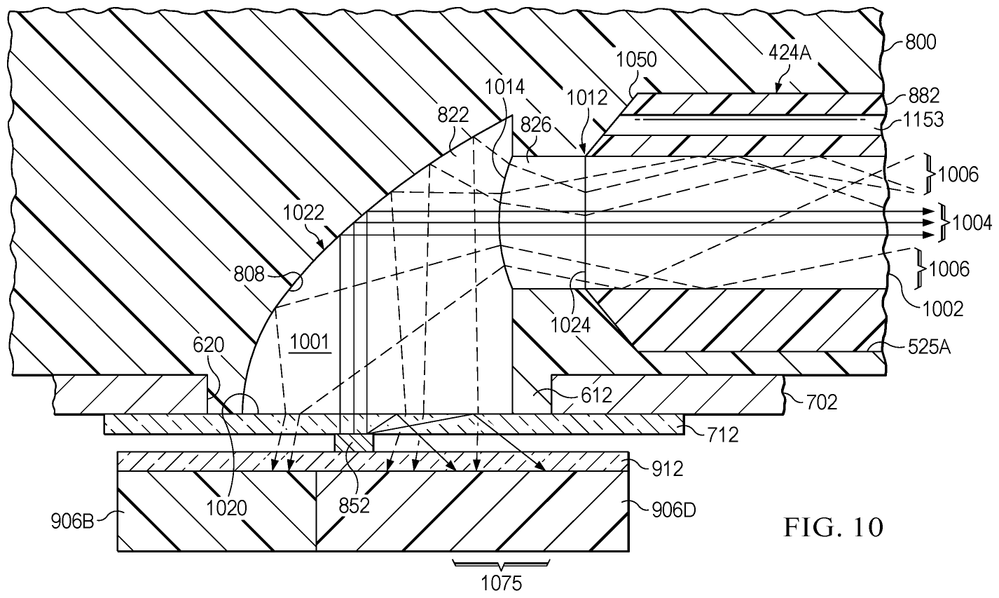

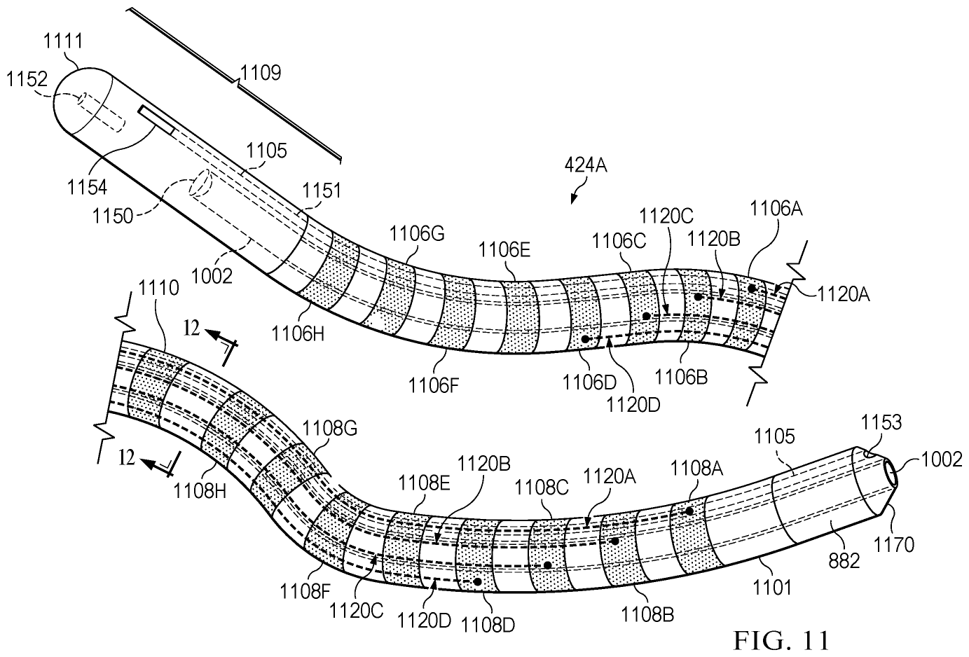

Referring then to FIG. 10, a partial cross section view of IPG 401, in use, will be further described. It should be understood that each parabolic redirector, die stack and mating components in the header and lead are structurally and functionally identical, hence only one set will be described here as an example.

Parabolic redirector 822 is further comprised of parabolic body 1001 and integral lens 826. Parabolic body 1001 and integral lens 826 are preferably integrally formed from a crystal glass having an index of refraction of between about 1.46 and 1.68, similar to that of silicone resin.

Parabolic body 1001 includes parabolic surface 1022. Parabolic surface 1022 is preferably a paraboloid having a curvature designed to produce one focal point at the VCSEL and another focal point at the face of and aligned to the optical axis of the fiber.

Parabolic surface 1022 includes an exterior reflective coating, preferably vapor deposited silver. In other embodiments, parabolic surface 1022 is coated with a titanium dioxide compound. Preferably, parabolic surface 1022 is also polished.

Parabolic body 1001 further includes interface surface 1020 adjacent optical window 712. Interface surface 1020 is flat to within an acceptable optical tolerance. Preferably, an index matching material, such as an epoxy is resident adjacent the surface and the optical window to provide low reflection loss and part stability. In another preferred embodiment, interface surface 1020 may be positioned adjacent optical window 712 with an index matching gel and fixed in place with a suitable epoxy adhesive.

Parabolic redirector 822 is further comprised of integral lens 826. Integral lens 826 is a collimating lens and includes convex lens surface 1014 which is directed inward toward parabolic surface 1022. In one preferred embodiment, convex lens surface 1014 is fixed to parabolic body 1001 by a suitable index matching epoxy. In another preferred embodiment, the parabolic body and the integral lens are integrally formed. In this case, the convex lens surface is created by an appropriate density change between integral lens 826 and parabolic body 1001. Integral lens 826 includes interface surface 1024 adjacent fiber 1002. Interface surface 1024 is preferably ground flat within appropriate optical tolerances.

Header body 800 includes frustroconical receiver surface 1050 at the proximal end of lead retainer hole 525A. Interface surface 1024 is positioned parallel to and against fiber 1002 of lead 424A at optical interface 1012 and held in place by the interference between collet 882 and frustroconical receiver surface 1050 of lead retainer hole 525A.

Positioning block 612 forms an extension of the header body and serves to position the parabolic redirector. The position block is positioned within receiving window 620 of top section 702. The interface between receiving window 620 and positioning block 612 fixes the vertical optical axis of the parabolic redirector above VCSEL 852 and in position to reflect light from parabolic surface 1022 to optical axis 804 of fiber 1002 of lead 424A.

In use, VCSEL 852 produces laser light pulses 1004 which proceed vertically upward toward parabolic surface 1022. The parabolic surface reflects the light about 90 degrees from vertically upward to horizontal and aligns the light through the integral lens and into fiber 1002, along optical axis 804, exiting the fiber at its distal end toward the spinal cord. In all cases, the pulses from the VCSELs will be specularly reflected from the spinal cord, given its high multispectral albedo. However, blue, aqua, green or yellow pulses will be preferentially absorbed by surrounding tissues and by any hemoglobin, if present, while in the case of IR, the pulses will be preferentially reflected from these tissues. Upon reflecting from the spinal cord, receive rays 1006 are collected and retransmitted through fiber 1002 back to the parabolic redirector. Receive rays 1006 are not well aligned along the fiber and so impact integral lens 826 at various angles. Receive rays 1006 are expanded by convex lens surface 1014 where they are incident upon parabolic surface 1022, where they are collected and directed about 90 degrees from horizontal to vertically downward, toward optical window 712. Receive rays 1006 pass through optical window 712 and are incident on cover plate 912 where they are directed toward photodiodes 906B and 906D.

When VCSEL 852 is in operation, some small percentage of light pulses 1004 are back reflected from optical window 712. These back reflected pulses 1075 are incident immediately on cover plate 912, where they are transmitted to photodiode 906D. The back reflected pulses are converted to signals by the photodiode that are used by the main processor to normalize the power output of the VCSEL and correct for source power variation, as will be further described.

Photodiodes 906B and 906D convert receive rays 1006 into electrical signals which are communicated to optical signal processor 405 for further processing, as will be further described.

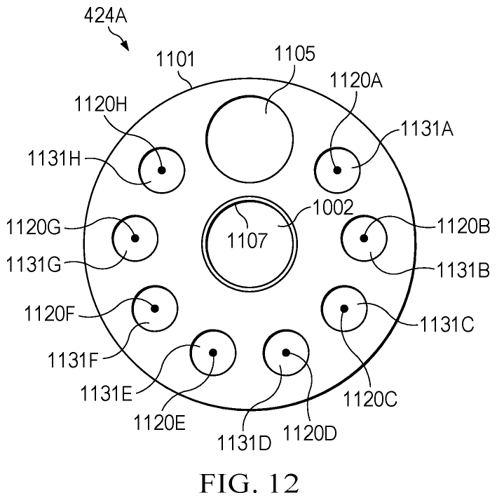

Referring then to FIGS. 11 and 12, lead 424A will be further described. Lead 424A, lead 424B, lead 422A and lead 422B are identical in structure and function. Only one will be described here, by way of example.

Lead 424A further comprises lead body 1101. Lead body 1101 is generally a flexible cylindrical extrusion distally terminated by transmission tip 1109 and proximally terminated by collet 882. In a preferred embodiment, the lead body is comprised of a flexible polymer, such as Pellethane 55D or similar biocompatible material. The lead body is preferably a multi-lumen extrusion having embedded and integrally formed components, as will be further described.

Transmission tip 1109 is an optically transparent cylinder fused to the distal terminus of the lead body. In a preferred embodiment, the transmission tip is a suitable optically transparent material such as a thermoplastic polyurethane. Transmission tip 1109 is terminated by semi-spherical cap 1111. In a preferred embodiment, semi-spherical cap 1111 and transmission tip 1109 are integrally formed. Transmission tip 1109 further includes embedded radiopaque marker 1152. Radiopaque marker 1152 is preferably titanium cylinder axially embedded adjacent semi-spherical cap 1111.

Fiber 1002 is positioned along the central optical axis of lead 424A and extends from collet 882 to concave lexicon 1150. The transmission tip is fused to fiber 1002. Fiber 1002 includes concave lexicon 1150 at its distal end. In a preferred embodiment, concave lexicon 1150 includes internally reflective coating such as titanium dioxide. Transmission tip 1109 further includes stylet channel terminus 1151. In a preferred embodiment, stylet channel terminus 1151 is a cylindrical opening. Stylet stop 1154 is positioned at the distal end of stylet channel terminus 1151. Stylet stop 1154 is preferably a titanium cylinder.

Stylet channel 1105 is coaxial with and extends from stylet channel terminus 1151 to stylet channel opening 1153, in collet 882. The stylet channel is a cylindrical cavity which runs parallel with the optical fiber and serves the purpose of housing a guide stylet for use during placement of the lead during surgery. In preferred embodiment, stylet channel 1105 is lined with a polytetrafluoroethylene (PTFE) lining 1107, which extends the length of the lead body. The low surface friction afforded by the lining facilitates insertion of the stylet during surgery.

Lead body 1101 further supports metallic anchor 1110 positioned at its proximal end. The metallic anchor is generally cylindrical and is permanently affixed to the exterior of the lead body.

Adjacent metallic anchor 1110, are eight cylindrical proximal metallic contacts, 1108A, 1108B, 1108C, 1108D, 1108E, 1108F, 1108G, and 1108H are fixed to the exterior of the lead body at even axial distances and are positioned to electrically contact the toroidal contacts in the header assembly.

Likewise, eight cylindrical metallic electrodes 1106A, 1106B, 1106C, 1106D, 1106E, 1106F, 1106G, and 1106H are fixed to the distal end of the lead body. The metallic electrodes are each permanently fixed to the exterior surface of the lead body, at equal axial distances.

The lead body further comprises eight radially oriented lumens, 1131A, 1131B, 1131C, 1131D, 1131E, 1131F, 1131G, and 1131H. Conductors 1120A, 1120B, 1120C, 1120D, 1120E, 1120F, 1120G and 1120H are integrally formed in the lumens and extend from their respective proximal contacts to their respective distal electrodes. In a preferred embodiment, the conductors are comprised of MP35N, or another conductive material similarly resistant to corrosion. Each of the conductors to connect exactly one proximal contact with exactly one paired metallic electrode.

In a preferred embodiment, fiber 1002 and the conductors are integrally formed into the lead body during manufacture.

In a preferred embodiment, collet 882 is formed from a suitable ceramic or crystal sapphire material. Collet 882 includes frustoconical surface 1170 at its proximal end. The frustoconical surface mates with an identical frustoconical receiver surface 1050 in header body 800 and aids in positioning fiber 1002 against optical interface 1012, and in radially compressing the fiber to aid in optical alignment with the parabolic redirector.

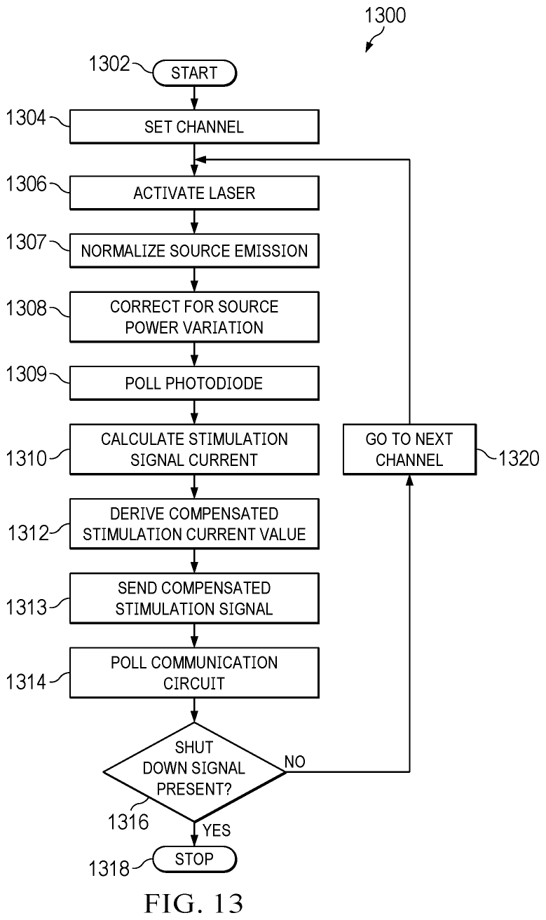

Referring to FIG. 13, method of IPG operation 1300 will be further described. In preferred embodiment, the method is carried out by programming instructions, which are resident in onboard memory of main processor 407.

At step 1302, the method begins.

At step 1304, the main processor sets an initial channel for operation. In a preferred embodiment, an initial channel includes one of the group of four leads 422A, 422B, 424A and 424B. In other preferred embodiments, the initial channel includes one of the group of only two or three of the leads.

In each case, the main processor addresses only eight electrodes on one lead at any one time. Each of the eight electrodes on the lead selected may be individually addressed by the main processor with a different current level for the stimulation signal.

At step 1306, main processor 407 activates the VCSEL for the specified channel. Preferably, the VCSEL produces a light pulse in the IR wavelength range. A small percentage of the light pulse is immediately back reflected by the optical window. The majority of the light pulse is sent to the base of the parabolic redirector where it passes to the parabolic surface which turns the light about 90 degrees from vertical to the horizontal and focuses it along the optical axis of the optical fiber for the chosen lead where it traverses the lead and exits from the concave lexicon through the transmission tip. The transmitted ray then is incident on the spinal cord, hemoglobin and other surrounding tissues, where it is reflected and received by the fiber at the concave lexicon. The received ray is transmitted down the fiber returning to the parabolic redirector where it is turned about 90 degrees, from the horizontal to vertically downward, and focused on the die stack.

At step 1307, main processor 407 uses the back reflected pulse to normalize the source emission, as will be further described.

At step 1308, the main processor uses the back reflected pulse to correct for power variation over time, will be further described.

At step 1309, the main processor polls the photodiode to obtain a feedback signal.

At step 1310, the main processor calculates a stimulation signal based on the signal from the photodiode. The stimulation signal is preferably generated according to a table, accurately disclosed in U.S. Pat. No. 9,550,063 to Wolf II, incorporated herein by reference. Of course, other stimulation routines may be used.

At step 1312, the main processor modifies the stimulation signal to compensate for the reduced optical feedback, according to the reflection compensation value, as will be further described.

At step 1313, the main processor activates the signal generator to send the compensated stimulation signal to the toroidal contacts for the lead for the chosen channel. The compensated stimulation signal is transmitted to the electrodes, which creates an electric field adjacent the target neurons at the spinal cord.

At step 1314, the main processor polls the communication circuit for a shutdown signal.

At step 1316, the main processor determines whether or not a shutdown signal is present. If not, the main processor moves to step 1320. If so, the main processor moves to step 1318.

At step 1320, the main processor advances to the next lead channel in the group of leads and returns to step 1306.

At step 1318, the main processor shuts down the routine and returns to a holding state.

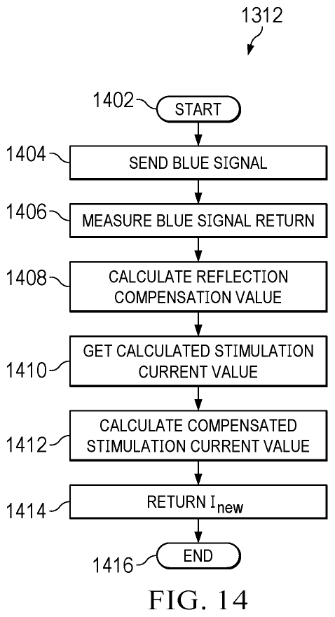

Referring then to FIG. 14, a preferred method of deriving a compensated stimulation current value of step 1312, will be further described.

At step 1402, the method begins.

At step 1404, the main processor activates the VCSEL to generate a second light pulse. Preferably, the VCSEL produces a light pulse in the blue wavelength range. However, other wavelengths may be used to long as they are a different wavelength than the first pulse. The blue laser signal proceeds through the parabolic redirector where it enters the fiber and is transmitted to the concave lexicon at the transmission tip. However, the blue light is preferentially absorbed by the surrounding tissues and hemoglobin. Most or all of the blue signal is absorbed by these tissues. As a result, the blue signal reflected is due primarily to Fresnel reflections in the optical system. Hence, the reflected blue signal represents optical system signal loss between the blue signal origination at the VCSEL and the blue signal reception at the photoreceiver. By monitoring the blue light reflections periodically, the optical system signal loss and increase in system noise over time can be recognized.

The optical system signal loss and noise are the same for each light signal regardless of wavelength. Hence, the optical signal system loss for the IR signal pulse will be the same as the optical signal system loss for the blue signal pulse. The preferred electro-optical controller generally increases stimulation current when the optical signal is decreased in intensity, based on the assumption that the spinal cord is farther away from the electrodes. So, as the optical losses in the system increase, the controller gradually increases the stimulation current. By decreasing the stimulation current by the same percentage that the optical losses increase, the optical degradation and Fresnel loss in the system can be compensated for, thereby providing the correct stimulation signal.

At step 1406, the return blue laser signal is measured by the photodiode which returns a current value to the main processor.

At step 1408, the main processor preferably calculates the percentage signal loss in the blue optical signal, known as the reflection compensation value, according to the following equation.

$$RCV = \frac{{I}_{t} - {I}_{r}}{{I}_{t}} \times 100$$

Where:
   RCV=percentage optical signal loss;
   Iₜ=the transmitted optical signal current to the VCSEL; and,
   Iᵣ=the received optical signal current from the photodiode.

At step 1410, the stimulation current value, “Iₛₜᵢₘ.”, is retrieved. Iₛₜᵢₘ is the current value calculated by the processor according to the correlation table between the return optical signal and the stimulation current, as previously described.

At step 1412, the stimulation current correction value, “I_{new}”, is preferably calculated according to the following equations.

RCV×Iₛₜᵢₘ=I_{new}
Where:
   Iₛₜᵢₘ=calculated stimulation current;
   RCV=percentage optical signal loss; and
   I_{new}=compensated stimulation current value.

At step 1414, the main processor returns the I_{new} value.

At step 1416, the method concludes.

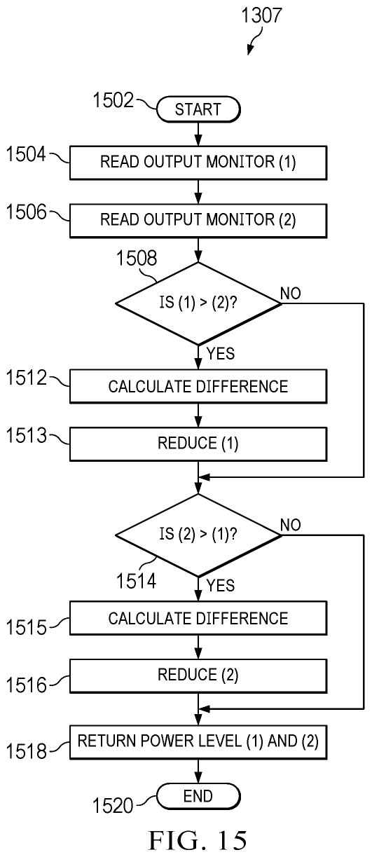

Referring then to FIG. 15, a preferred method of normalizing source emission of step 1307 will be further described. The method is the same for each die stack.

At step 1502, the method begins.

At step 1504, main processor 407 retrieves from memory the output of monitor photodiode one. In this example, photodiode one is photodiode 906C adjacent VCSEL 850.

At step 1506, the main processor reads the output value of monitor photodiode two. In this example, photodiode two is photodiode 906D adjacent VCSEL 852.

At step 1508, the main processor compares the output of monitor photodiode one and the output of monitor photodiode two. If the output of photodiode one is greater than the output of photodiode two, then the method moves to step 1512. If not, the method moves to step 1514.

At step 1512, the difference between the output of photodiode one and the output of photodiode two is calculated by subtracting the output of photodiode two from the output of photodiode one.

At step 1513, the drive current to photodiode one is reduced by the difference.

At step 1514, the output of photodiode two is compared to the output of photodiode one. If the output of photodiode two is greater than the output of photodiode one, then the method moves to step 1515. If not, the method moves to step 1518.

At step 1515, the difference between the output of photodiode two and the output of photodiode one is calculated by subtracting the output of photodiode one from the output of photodiode two.

At step 1516, the drive current to photodiode two is reduced by the difference.

At step 1518, the method returns the normalized power levels for photodiode one and photodiode two.

At step 1520, the method ends.

In a preferred embodiment, the differences are calculated as percentages which can be directly applied to the photodiode current to normalize the source of emission.

In one preferred embodiment, method 1307 is applied between the two VCSELs in one photodiode stack, such as VCSEL 850 and VCSEL 852. However, in other embodiments, normalization may take place over all four VCSELs in the system, so that each produces the same level of source emission.

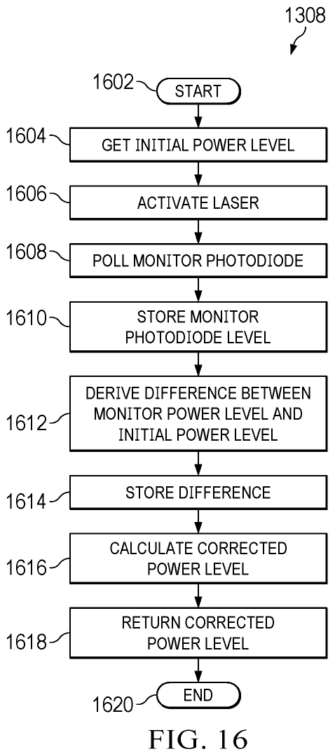

Referring then to FIG. 16, a preferred method of correcting for source power variation of step 1308 will be further described.

At step 1602, the method begins. Ideally, the method takes place only periodically, such as once a week, so as to conserve system power usage.

At step 1604, the main processor retrieves the initial power output level of the VCSEL from memory. Preferably, the initial power output level is measured by measuring the current from the monitor photodiode adjacent the VCSEL when the VCSEL is initially activated during system startup.

At step 1606, the main processor activates the chosen VCSEL.

At step 1608, the main processor polls the monitor photodiode for the chosen VCSEL.

At step 1610, the main processor stores the monitor photodiode power level.

At step 1612, the main processor derives a difference between the monitor photodiode power level and the initial monitor photodiode power level. According to the following equation:

PL_{I}−PLₘ=Δ
Where:
   Δ=the difference;
   PL_{I}=the initial power level; and
   PLₘ the measured power level.

At step 1614, the difference is stored as a percentage according to the following equation:

$${PL}_{DIFF} = \frac{{PL}_{I} - {PL}_{m}}{100}$$

Where:
   PL_{DIFF}=the percentage power level difference;
   PL_{I}=the initial power level; and
   PLₘ=the measured power level.

At step 1616, the corrected power level is calculated according to the following equation:

I₁×PL_{DIFF}=I₂
Where:
   I₁=the initial current level;
   PL_{DIFF}=the percentage difference; and
   I₂=the new power level.

At step 1618, the main processor returns the new power level.

At step 1620, the method concludes.

1. An implantable pulse generator system comprising:
a case;
a lead retainer hole, longitudinally positioned in the case, having an optical axis;
a parabolic redirector, having a first interface surface perpendicular to a second interface surface connected by a parabolic surface, focused on the optical axis;
a die stack, adjacent the parabolic redirector, perpendicular to the optical axis and parallel to the second interface surface;
a laser, fixed on the die stack, directed vertically toward to the second interface surface;
a photo receiver, positioned around the laser and parallel to the second interface surface; and
a processor, having a memory, operatively connected to the laser and the photo receiver.

2. The implantable pulse generator system of claim 1, wherein the laser is a VCSEL.

3. The implantable pulse generator system of claim 1, wherein the parabolic redirector includes a collimating lens centered on the optical axis.

4. The implantable pulse generator system of claim 1, wherein the laser is fixed on the die stack by an optical window.

5. The implantable pulse generator system of claim 4, wherein the laser is electrically connected to the processor by a metallic trace, fixed on the optical window.

6. The implantable pulse generator system of claim 1, wherein the case further comprises:
a metallic first section forming a header bay;
a header, containing the lead retainer hole, fixed in the header bay; and
a ceramic second section, hermetically sealed to the metallic first section.

7. The implantable pulse generator system of claim 1, further comprising:
a set of toroidal electrical contacts, fixed in the lead retainer hole, axially aligned with the optical axis.

8. The implantable pulse generator system of claim 7, further comprising:
a flexible lead, fixed in the lead retainer hole;
a centrally disposed optical fiber, integrally formed in the flexible lead, coaxial with the optical axis;
a set of cylindrical contacts, fixed on an exterior surface of the flexible lead, electrically connected to the set of toroidal electrical contacts; and
a set of cylindrical electrodes, fixed on the exterior surface of the flexible lead, electrically connected to the set of cylindrical contacts.

9. The implantable pulse generator system of claim 8, wherein the flexible lead further comprises:
a longitudinal stylet lumen, radially disposed adjacent and parallel to the centrally disposed optical fiber.

10. The implantable pulse generator system of claim 9, wherein the longitudinal stylet lumen terminates distally in a stylet stop.

11. The implantable pulse generator system of claim 9, wherein the flexible lead further comprises:
a transparent optical transmission tip, integrally formed with the centrally disposed optical fiber.

12. The implantable pulse generator system of claim 11, wherein the transparent optical transmission tip further comprises a centrally disposed radiopaque marker.

13. The implantable pulse generator system of claim 8, further comprising:
a set of instructions, resident in the memory, that when executed cause the implantable pulse generator system to:
generate a first transmit ray, at a first wavelength, from the laser;
send the first transmit ray through the parabolic redirector down the centrally disposed optical fiber;
receive a first receive ray, from the centrally disposed optical fiber, through the parabolic redirector, incident on the photo receiver;
generate a variation variable from the first receive ray;
create a modulated stimulation signal based on the variation variable; and
send the modulated stimulation signal to the set of cylindrical electrodes.

14. The implantable pulse generator system of claim 13, wherein the set of instructions comprises further instructions, resident in the memory, that when executed cause the implantable pulse generator system to:
generate a second transmit ray, of a second wavelength, from the laser;
send the second transmit ray through the parabolic redirector down the centrally disposed optical fiber;
receive a second receive ray, from the centrally disposed optical fiber, through the parabolic redirector incident on the photo receiver;
generate a compensation value from the second receive ray; and
alter the modulated stimulation signal based on the compensation value.

15. An implantable pulse generator system comprising:
a case;
a first header bay, formed in the case;
a second header bay, diametrically disposed to the first header bay, formed in the case;
a first header assembly, fixed in the first header bay, having a first lead retainer channel and a second lead retainer channel;
a second header assembly, fixed in the second header bay, having a third lead retainer channel and a fourth lead retainer channel;
a first electro-optical lead, having a first optical axis, positioned in the first lead retainer channel;
a second electro-optical lead, having a second optical axis, positioned in the second lead retainer channel;
a third electro-optical lead, having a third optical axis, positioned in the third lead retainer channel;
a fourth electro-optical lead, having a fourth optical axis, positioned in the fourth lead retainer channel;
a first parabolic redirector, centered on the first optical axis, optically coupled to the first electro-optical lead;
a second parabolic redirector, centered on the second optical axis, optically coupled to the second electro-optical lead;
a third parabolic redirector, centered on the third optical axis, optically coupled to the third electro-optical lead;
a fourth parabolic redirector, centered on the fourth optical axis, optically coupled to the fourth electro-optical lead;
a first die stack, having a first perpendicularly oriented laser, surrounded by a first photodiode, optically coupled to the first parabolic redirector;
the first die stack, having a second perpendicularly oriented laser, surrounded by a second photodiode, optically coupled to the second parabolic redirector;
a second die stack, having a third perpendicularly oriented laser, surrounded by a third photodiode, optically coupled to the third parabolic redirector; and
the second die stack, having a fourth perpendicularly oriented laser, surrounded by a fourth photodiode, optically coupled to the fourth parabolic redirector.

16. The implantable pulse generator system of claim 15 wherein the first electro-optical lead further comprises:
an optical fiber, positioned coaxially with the first optical axis;
a set of electrical contacts, fixed on a proximal surface of the first electro-optical lead; and
a set of electrodes, fixed on a distal surface of the first electro-optical lead, electrically connected to the set of electrical contacts.

17. The implantable pulse generator system of claim 16, wherein the first header bay further comprises:
a set of fixed contacts, rigidly positioned in the first lead retainer channel, and operatively connected to an electro-optical signal generator;
wherein the electro-optical signal generator is programmed to:
send a first transmit ray, of a first wavelength, from the first perpendicularly oriented laser into the first parabolic redirector and the optical fiber;
receive a first return signal from the first photodiode, based on a first receive ray;
generate a stimulation signal based on the first return signal; and
send the stimulation signal to the set of fixed contacts, for transmission to the set of electrodes.

18. The implantable pulse generator system of claim 17, wherein the electro-optical signal generator is further programmed to:
send a second transmit ray, of a second wavelength, from the first perpendicularly oriented laser into the first parabolic redirector and the first electro-optical lead;
receive a second receive ray at the first photodiode;
generate a compensation value from the second receive ray; and
modify the stimulation signal based on the compensation value.

19. The implantable pulse generator system of claim 17, wherein:
the first die stack further comprises a second photodiode;
wherein the electro-optical signal generator is further programmed to:
normalize a first supply current to the first photodiode and a second supply current to the second photodiode.

20. The implantable pulse generator system of claim 19, wherein the step of normalizing further comprises:
reducing the first supply current if the first supply current is greater than the second supply current; and
reducing the second supply current if the second supply current is greater than the first supply current.

21. The implantable pulse generator system of claim 19, wherein the electro-optical signal generator is further programmed to:
correct for a time based variation in the first supply current.

22. The implantable pulse generator system of claim 21, wherein the step of correcting further comprises:
deriving a difference between an initial supply current to the first photodiode and the first supply current.

23. The implantable pulse generator system of claim 18, wherein:
the first wavelength is between about 700 nanometer and about 800 nanometers; and
the second wavelength is between about 400 nanometers and about 500 nanometers.

24. The implantable pulse generator system of claim 18, wherein:
the first wavelength is between about 700 nanometer and about 800 nanometers; and
the second wavelength is between about 520 nanometers and about 532 nanometers.

25. The implantable pulse generator system of claim 15, wherein the first parabolic redirector further comprises:
a collimating lens centered on the first optical axis; and
a parabolic surface, for reflecting a transmit ray toward the first electro-optical lead and a receive ray toward the first photodiode.

26. The implantable pulse generator system of claim 25, wherein the parabolic surface includes a reflective coating.

27. The implantable pulse generator system of claim 15, further comprising:
a transparent cover plate, between the first perpendicularly oriented laser and the first die stack, positioning the first perpendicularly oriented laser adjacent the first parabolic redirector.

28. The implantable pulse generator system of claim 27, further comprising:
a window, sealed to the case, between the first perpendicularly oriented laser and the first parabolic redirector.

29. A pulse generator lead comprising:
a flexible lead body;
a centrally disposed optical fiber, integrally formed in the flexible lead body, coaxial with an optical axis;
a set of cylindrical contacts, fixed on an exterior surface of the flexible lead body, electrically connected to a set of toroidal electrical contacts;
a set of cylindrical electrodes, fixed on the exterior surface of the flexible lead body, electrically connected to the set of cylindrical contacts, by a set of wires integrally formed in the flexible lead body; and
a longitudinal stylet lumen, radially disposed adjacent and parallel to the centrally disposed optical fiber.

30. The pulse generator lead of claim 29, wherein the longitudinal stylet lumen terminates distally in a stylet stop cylinder.

31. The pulse generator lead of claim 30, further comprising:
a transparent optical transmission tip, integrally formed with the centrally disposed optical fiber.

32. The pulse generator lead of claim 31, wherein the transparent optical transmission tip further comprises a centrally disposed radiopaque marker.
